SEQUENCE LISTING (83 sequences)

SEQ ID NO: 1 - DNA, 483 bp, synthetic construct
  source 1..483: mol_type = other DNA
atcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 2 - DNA, 402 bp, synthetic construct
  source 1..402: mol_type = other DNA
aataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 3 - DNA, 483 bp, synthetic construct
  source 1..483: mol_type = other DNA
atcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgac

SEQ ID NO: 4 - DNA, 69 bp, synthetic construct
  source 1..69: mol_type = other DNA
atgggtcgggggctgctcaggggcctgtggccgctgcacatcgtcctgtggacgcgtatcgccagcacg

SEQ ID NO: 5 - DNA, 96 bp, synthetic construct
  source 1..96: mol_type = other DNA
atgcacagtcaagggcggggttgcaacaacacaaaacaaaacaaaacttccggacttcgacctgcagctgagaagaacatctcgcaaagcggcgtt

SEQ ID NO: 6 - DNA, 69 bp, synthetic construct
  source 1..69: mol_type = other DNA
atgggtcgggggctgctccggggcctgtggccgctgcatatcgtcctgtggacgcgcatcgccagcacg

SEQ ID NO: 7 - protein, 23 aa, synthetic construct
MGRGLLRGLWPLHIVLWTRIAST

SEQ ID NO: 8 - protein, 32 aa, synthetic construct
MHSQGRGCNNTKQNKTSGLRPAAEKNISQSGV

SEQ ID NO: 9 - DNA, 552 bp, synthetic construct
  source 1..552: mol_type = other DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 10 - DNA, 498 bp, synthetic construct
  source 1..498: mol_type = other DNA
atgcacagtcaagggcggggttgcaacaacacaaaacaaaacaaaacttccggacttcgacctgcagctgagaagaacatctcgcaaagcggcgttaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 11 - DNA, 471 bp, synthetic construct
  source 1..471: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 12 - DNA, 552 bp, synthetic construct
  source 1..552: mol_type = other DNA
atgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgac

SEQ ID NO: 13 - protein, 161 aa, synthetic construct
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 14 - protein, 161 aa, synthetic construct
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 15 - protein, 127 aa, synthetic construct
NNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 16 - protein, 184 aa, synthetic construct
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 17 - protein, 159 aa, synthetic construct
MHSQGRGCNNTKQNKTSGLRPAAEKNISQSGVNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 18 - protein, 184 aa, synthetic construct
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 19 - protein, 150 aa, synthetic construct
MGRGLLRGLWPLHIVLWTRIASTNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 20 - DNA, 711 bp, synthetic construct
  source 1..711: mol_type = other DNA
cccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 21 - DNA, 696 bp, synthetic construct
  source 1..696: mol_type = other DNA
cccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 22 - protein, 237 aa, synthetic construct
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 23 - protein, 232 aa, synthetic construct
PRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - DNA, 1113 bp, synthetic construct
  source 1..1113: mol_type = other DNA
aataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 25 - DNA, 1182 bp, synthetic construct
  source 1..1182: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 26 - protein, 371 aa, synthetic construct
NNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPDLLLVIFQPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 27 - protein, 394 aa, synthetic construct
MGRGLLRGLWPLHIVLWTRIASTNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPDLLLVIFQPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - DNA, 627 bp, synthetic construct
  source 1..627: mol_type = other DNA
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcc

SEQ ID NO: 29 - DNA, 633 bp, synthetic construct
  source 1..633: mol_type = other DNA
atgggctgggccgaggccgggttcgagcacctgggactgtgggtccctgtgctggctgtgcttttgctggaagcctgccgggcacatccgatccctgactccagccccctcctacaatttggaggtcaagttcgacagcggtacctctacaccgacgatgcccaggagacagaggcccacctagagatcagggccgatggcacagtggtgggggctgcccgccagagccctgaaagtctcctggagctgaaagccctaaagccaggggtcattcaaatcttgggagtcaaaacatccaggttcctgtgccagggcccagatgggacactatatggctcgctccatttcgaccctgtggcctgcagtttccgagaactgcttcttgaggatgggtacaacatctaccactccgagacccttggtctcccgcttcgcctgcgcccccacaactccgcataccgggacttggcaccccgcgggcctgcccgcttcctgccactgccaggcctgcttccagcacccccagagcctccagggatcctggccccggagcctcctgacgtgggctcctcggaccctctgagcatggtggggccttcacagggccggagtcccagctatgcttcctgatag

SEQ ID NO: 30 - DNA, 627 bp, synthetic construct
  source 1..627: mol_type = other DNA
atggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcc

SEQ ID NO: 31 - DNA, 633 bp, synthetic construct
  source 1..633: mol_type = other DNA
atggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctga

SEQ ID NO: 32 - protein, 209 aa, synthetic construct
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 33 - protein, 209 aa, synthetic construct
MGWAEAGFEHLGLWVPVLAVLLLEACRAHPIPDSSPLLQFGGQVRQRYLYTDDAQETEAHLEIRADGTVVGAARQSPESLLELKALKPGVIQILGVKTSRFLCQGPDGTLYGSLHFDPVACSFRELLLEDGYNIYHSETLGLPLRLRPHNSAYRDLAPRGPARFLPLPGLLPAPPEPPGILAPEPPDVGSSDPLSMVGPSQGRSPSYAS

SEQ ID NO: 34 - protein, 210 aa, synthetic construct
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 35 - DNA, 1153 bp, synthetic construct
  source 1..1153: mol_type = other DNA
gggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggt

SEQ ID NO: 36 - DNA, 231 bp, synthetic construct
  source 1..231: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 37 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 38 - DNA, 393 bp, synthetic construct
  source 1..393: mol_type = other DNA
atcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtg

SEQ ID NO: 39 - DNA, 154 bp, synthetic construct
  source 1..154: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 40 - DNA, 504 bp, synthetic construct
  source 1..504: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 41 - DNA, 100 bp, synthetic construct
  source 1..100: mol_type = other DNA
ccaaaatcaacgggactttccaaaatgtcgtaataaccccgccccgttgacgcaaatgggcggtaggcgtgtacggtgggaggtctatataagcagagct

SEQ ID NO: 42 - DNA, 1873 bp, synthetic construct
  source 1..1873: mol_type = other DNA
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 43 - DNA, 913 bp, synthetic construct
  source 1..913: mol_type = other DNA
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcacc

SEQ ID NO: 44 - DNA, 1246 bp, synthetic construct
  source 1..1246: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 45 - DNA, 1061 bp, synthetic construct
  source 1..1061: mol_type = other DNA
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 46 - DNA, 953 bp, synthetic construct
  source 1..953: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 47 - DNA, 82 bp, synthetic construct
  source 1..82: mol_type = other DNA
gtaagggtttaagggatggttggttggtggggtattaatgtttaattacctggagcacctgcctgaaatcactttttttcag

SEQ ID NO: 48 - DNA, 476 bp, synthetic construct
  source 1..476: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 49 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 50 - DNA, 248 bp, synthetic construct
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 51 - DNA, 132 bp, synthetic construct
  source 1..132: mol_type = other DNA
gctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 52 - DNA, 510 bp, synthetic construct
  source 1..510: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 53 - DNA, 387 bp, synthetic construct
  source 1..387: mol_type = other DNA
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 54 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 55 - DNA, 54 bp, synthetic construct
  source 1..54: mol_type = other DNA
gagggccgcggcagcctgctgacctgcggcgacgtggaggaaaaccccggcccc

SEQ ID NO: 56 - DNA, 59 bp, synthetic construct
  source 1..59: mol_type = other DNA
ggatctggcgccaccaacttctctctgctgaagcaggccggcgacgtggaggagaaccc

SEQ ID NO: 57 - DNA, 581 bp, synthetic construct
  source 1..581: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagccacc

SEQ ID NO: 58 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 59 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 60 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 61 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 62 - DNA, 2214 bp, synthetic construct
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 63 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 64 - protein, 601 aa, synthetic construct
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 65 - protein, 535 aa, synthetic construct
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 66 - DNA, 3196 bp, synthetic construct
  source 1..3196: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgagtgatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaataataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 67 - DNA, 4611 bp, synthetic construct
  source 1..4611: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatgaattctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgagtgatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaataataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagccacgcgtgaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacatgactagtcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 68 - DNA, 2640 bp, synthetic construct
  source 1..2640: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcctaataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 69 - DNA, 4596 bp, synthetic construct
  source 1..4596: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctaccggtgaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtgttactgtccaatggaattctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcctaataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagccacgcgtgtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaagtgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcactagtaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 70 - DNA, 235 bp, synthetic construct
  source 1..235: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgaca

SEQ ID NO: 71 - DNA, 1247 bp, synthetic construct
  source 1..1247: mol_type = other DNA
atgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaa

SEQ ID NO: 72 - DNA, 1178 bp, synthetic construct
  source 1..1178: mol_type = other DNA
atcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaa

SEQ ID NO: 73 - protein, 415 aa, synthetic construct
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 74 - protein, 392 aa, synthetic construct
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 75 - DNA, 169 bp, synthetic construct
  source 1..169: mol_type = other DNA
tttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 76 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 77 - DNA, 140 bp, synthetic construct
  source 1..140: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 78 - DNA, 2939 bp, synthetic construct
  source 1..2939: mol_type = other DNA
gggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgcggcctgccaccatgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggagggagtggtgggtccgattacaaagatcacgatggggactataaagatcacgacatcgactataaggatgacgatgataaatgaataagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 79 - DNA, 3379 bp, synthetic construct
  source 1..3379: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattccttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgcggcctgccaccatgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggagggagtggtgggtccgattacaaagatcacgatggggactataaagatcacgacatcgactataaggatgacgatgataaatgaataagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 80 - DNA, 1319 bp, synthetic construct
  source 1..1319: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagcggccgctcgccaccatggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctgaagcgggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 81 - DNA, 1738 bp, synthetic construct
  source 1..1738: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattccaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagcggccgctcgccaccatggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctgaagcgggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 82 - DNA, 696 bp, synthetic construct; identical to SEQ ID NO: 21
  source 1..696: mol_type = other DNA

SEQ ID NO: 83 - protein, 232 aa, synthetic construct; identical to SEQ ID NO: 23

(19) United States
(12) Patent Application Publication
(10) Pub. No.: US 2026/0102513 A1
(43) Pub. Date: Apr. 16, 2026

(54) GENE THERAPY METHODS FOR ARRHYTHMOGENIC CARDIOMYOPATHY

(71) Applicant: REJUVENATE BIO, San Diego, CA (US)

(72) Inventors: Noah Davidsohn, Brookline, MA (US); Dan Oliver, San Diego, CA (US)

(21) Appl. No.: 19/114,132

(22) PCT Filed: Sep. 21, 2023
(86) PCT No.: PCT/US23/74747

§ 371 Date: Mar. 21, 2025

Related U.S. Application Data
(60) Provisional application No. 63/376,474, filed on Sep. 21, 2022.

(51) Int. Cl. A61K 48/00; A61K 38/17; A61K 38/18; A61P 9/06; C12N 15/86

(52) U.S. Cl. CPC A61K 48/005; A61K 38/179; A61K 38/1825; A61P 9/06; C12N 15/86; C12N 2750/14143; C12N 2830/48; C12N 2830/50

(57) ABSTRACT

The present disclosure provides methods of treating an arrhythmogenic cardiomyopathy (ACM) in a subject. The methods generally comprise administering to the subject a gene therapy comprising a first nucleic acid encoding soluble transforming growth factor beta receptor 2 (sTGFbR2) and a second nucleic acid encoding fibroblast growth factor 21 (FGF21).

### RELATED APPLICATIONS

This application claims priority to U.S. Provisional Patent Application Ser. No. 63/376,474, filed Sep. 21, 2022, the entire disclosure of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Sep. 19, 2023, is named “203409_SL.xml” and is 131,990 bytes in size).

### BACKGROUND

Arrhythmogenic cardiomyopathy (ACM) is an inherited heart muscle disorder characterized by redistribution of junctional proteins, arrhythmias, and progressive myocardial injury. Pathological features of ACM include loss of myocytes and fibrofatty replacement of right ventricular myocardium. Biventricular involvement is often observed. ACM is a cell junction cardiomyopathy, typically caused by genetic abnormalities of cardiac desmosomes, which results in the detachment of myocytes and affects intracellular signal transduction. The prevalence of ACM in the United States is estimated to be one in five thousand people. The rate of prevalence can depend on geography, as cases reach one in one thousand people in certain regions of the world such as Northem Italy. There are three phenotypic variants of ACM: including the dominant-right variant, also known as the classic arrhythmogenic right ventricular cardiomyopathy (ARVC) phenotype, characterized by the predominant right ventricle involvement, with no left ventricle abnormalities; the biventricular disease variant, characterized by the involvement of both right and left ventricles; and the dominant-left variant, also known as arrhythmogenic left ventricular cardiomyopathy (ALVC), characterized by left ventricle involvement with no right ventricle abnormalities.

There is no cure for ACM. Treatment of ACM is directed toward symptomatic relief of primarily the electrophysiological defects. As such, existing therapies for ACM include use of anti-arrhythmic drugs that may transition into surgical interventions, including the implantation of cardioverter defibrillators and cardiac catheter ablation, for patients who become refractory to anti-arrhythmic therapies.

Accordingly, there is a need in the art for methods for treating arrhythmogenic cardiomyopathy.

### SUMMARY

Provided herein are methods of treating an arrhythmogenic cardiomyopathy (ACM) in a subject. The methods generally comprise administering to the subject a gene therapy comprising a first nucleic acid encoding soluble transforming growth factor beta receptor 2 (sTGFβR2) and a second nucleic acid encoding fibroblast growth factor 21 (FGF21).

Accordingly, in one aspect, the instant disclosure provides a method of treating an arrhythmogenic cardiomyopathy (ACM) in a subject, comprising administering to the subject a gene therapy comprising a first nucleic acid encoding soluble transforming growth factor beta receptor (sTGFβR2) and a second nucleic acid encoding fibroblast growth factor 21 (FGF21).

In certain embodiments, the ACM is selected from the group consisting of arrhythmogenic right ventricular cardiomyopathy (ARVC), arrhythmogenic left ventricular cardiomyopathy (ALVC), and biventricular arrhythmogenic cardiomyopathy. In certain embodiments, the ACM is ARVC.

In certain embodiments, the subject is a mammal. In certain embodiments, the subject is a human.

In certain embodiments, the method further comprises determining a base level of fractional shortening (FS) and/or left ventricular ejection fraction (LVEF) in the subject prior to administration of the gene therapy. In certain embodiments, the treatment results in a reduction in the level of FS and/or LVEF in the subject at a duration after administration of the gene therapy, as compared to the base level of FS and/or LVEF in the subject.

In certain embodiments, the subject has arrhythmia. In certain embodiments, the method further comprises determining a base level of arrhythmia in the subject prior to administration of the gene therapy. In certain embodiments, the treatment results in a reduction in the level of arrhythmia in the subject at a duration after administration of the gene therapy, as compared to the base level of arrhythmia in the subject.

In certain embodiments, the subject has been administered an effective amount of one or more therapeutic agents for the treatment of an ACM. In certain embodiments, the method further comprises administering to the subject an effective amount of one or more therapeutic agents for the treatment of an ACM.

In certain embodiments, the one or more therapeutic agents for the treatment of an ACM is selected from the group consisting of an angiotensin-converting enzyme (ACE) inhibitor, an angiotensin II receptor blocker, an anti-arrhythmia agent, a beta blocker, a blood thinner, and a diuretic.

In certain embodiments, the gene therapy is administered intravenously.

In certain embodiments, the ACM is PKP2 mutation-associated ACM. In certain embodiments, the ACM is DSG2 mutation-associated ACM. In certain embodiments, the ACM is DSP mutation-associated ACM. In certain embodiments, the ACM is DSC mutation-associated ACM. In certain embodiments, the ACM is DSC2 mutation-associated ACM. In certain embodiments, the ACM may have more than one gene variant involved, including PKP2 and/or DSG2 and/or DSP.

In certain embodiments, the ACM is a genetically elusive ACM.

In certain embodiments, the first nucleic acid comprises a first transcriptional regulatory element operably linked to the sTGFBR2 coding sequence. In certain embodiments, the sTGFBR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 1, 2, 3, 24, or 72. In certain embodiments, the sTGFBR2 coding sequence further comprises a heterologous or an innate secretion signal sequence, wherein the signal sequence is encoded by a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 4, 5, or 6. In certain embodiments, the sTGFBR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 9, 10, 11, 12, 25, or 71. In certain embodiments, the sTGFBR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 13, 14, 15, 26, or 74. In certain embodiments, the sTGFBR2 coding sequence further encodes a secretion signal sequence having at least 85%, 86%, 87%, 88%, 89%, 90%, 91%, 92%, 93%, 94%, 95%, 96%, 97%, 98%, 99%, or 100% sequence identity to SEQ ID NO: 7 or 8. In certain embodiments, the sTGFBR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, 17, 18, 19, 27, or 73.

In certain embodiments, the second nucleic acid comprises a second transcriptional regulatory element operably linked to the FGF21 coding sequence. In certain embodiments, the FGF21 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 28, 29, 30, or 31. In certain embodiments, the FGF21 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 32, 33, or 34.

In certain embodiments, the first and second transcriptional regulatory elements are each selected from the group consisting of one or more ApoE binding sites, an hAAT promoter, and an EF-1α promoter or variant thereof. In certain embodiments, the first and second transcriptional regulatory elements each comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 34, 36, 38, 39, 40, and/or 48.

In certain embodiments, the first and second nucleic acid each further comprises a post-transcriptional regulatory element. In certain embodiments, the post-transcriptional regulatory element comprises a polyadenylation signal and/or WPRE sequence. In certain embodiments, the polyadenylation signal is an SV40 polyadenylation signal. In certain embodiments, the WPRE sequence is a WPRE3 sequence. In certain embodiments, the post-transcriptional regulatory element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 50, 51, 70, or 75.

In certain embodiments, the first nucleic acid is comprised within a first vector, and the second nucleic acid is comprised within a second vector. In certain embodiments, the first vector and/or the second vector is each a viral vector, optionally wherein each is independently selected from the group consisting of adeno-associated virus (AAV), adenovirus, retrovirus, orthomyxovirus, paramyxovirus, papovavirus, picomavirus, lentivirus, herpes simplex virus, vaccinia virus, pox virus, and alphavirus.

In certain embodiments, the first vector is an AAV vector comprised within a first recombinant AAV (rAAV), wherein the first rAAV comprises an AAV capsid comprising an AAV capsid protein; and a first rAAV genome; and/or the second vector is an AAV vector comprised within a second rAAV, wherein the second rAAV comprises an AAV capsid comprising an AAV capsid protein; and a second rAAV genome.

In certain embodiments, the gene therapy comprises: a first recombinant AAV (rAAV) comprising: an AAV capsid comprising an AAV capsid protein; and a first rAAV genome comprising a nucleic acid encoding the amino acid sequence set forth in SEQ ID NO: 26 or 73; and a second rAAV comprising: an AAV capsid comprising an AAV capsid protein; and a second rAAV genome comprising a nucleic acid encoding the amino acid sequence set forth in SEQ ID NO: 33 or 34.

In certain embodiments, the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 66 or 78. In certain embodiments, the first rAAV genome further comprises a 5′ inverted terminal repeat (5′ ITR) nucleotide sequence, and a 3′ inverted terminal repeat (3′ ITR) nucleotide sequence. In certain embodiments, the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77. In certain embodiments, the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 67 or 79.

In certain embodiments, the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 68 or 80. In certain embodiments, the second rAAV genome further comprises a 5′ inverted terminal repeat (5′ ITR) nucleotide sequence, and a 3′ inverted terminal repeat (3′ ITR) nucleotide sequence. In certain embodiments, the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77. In certain embodiments, the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 69 or 81.

In certain embodiments, the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof. In certain embodiments, the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

In certain embodiments, the first and the second nucleic acid are comprised within a vector, optionally wherein the first and the second nucleic acid are separated by a polycistronic element. In certain embodiments, the vector comprises: the first nucleic acid comprising a nucleic acid encoding the amino acid sequence set forth in SEQ ID NO: 26 or 73; and the second nucleic acid comprising a nucleic acid encoding the amino acid sequence set forth in SEQ ID NO: 33 or 34. In certain embodiments, the first and the second nucleic acid are separated by a polycistronic element. In certain embodiments, the polycistronic element is an IRES or 2A sequence. In certain embodiments, the polycistronic element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 55, 56, or 57.

In certain embodiments, the vector is an AAV vector comprised within a recombinant AAV (rAAV), wherein the rAAV comprises an AAV capsid comprising an AAV capsid protein; and a rAAV genome. In certain embodiments, the AAV capsid protein is derived from a clade A, clade B, clade C, clade D, clade E, clade F, clade G, clade H, clade I, AAVgo.1, AAV3, AAV4. AAV10, AAV11, AAV12, rh.32, rh32.33, rh.33, rh.34, BAAV, or AAV5 capsid protein, or an engineered variant thereof. In certain embodiments, the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

### BRIEF DESCRIPTION OF THE DRAWINGS

FIGS. 1A and 1B are graphs showing the level of sTGFβR2 (FIG. 1A) and FGF21 (FIG. 1B) expression over time in Dsg2^{mut/mut} (DSG2 Mut) or wild-type (WT) mice administered AAV8-sTGFβR2 and AAV8-FGF21 at a dose of 3E12 vg/kg and 3E11 vg/kg, respectively.

FIGS. 2A and 2B are graphs showing heart function, as measured by percent fractional shortening (% FS; FIG. 2A) and left ventricular rejection fraction (LVEF; FIG. 2B), of Dsg2^{mut/mut} (Dsg2 Mut) or wild-type (WT) mice administered AAV8-sTGFfPR2 and AAV8-FGF21 (GT) at a dose of 3E12 vg/kg and 3E11 vg/kg, respectively, or vehicle control. * indicates a p value of <0.05 determined by two-way ANOVA statistical test.

FIGS. 3A and 3B are graphs showing the level of FGF21 (FIG. 3A) and sTGF3R2 (FIG. 3B) expression in PKP2cKO mice administered AAV8-FGF21, AAV8-sTGFβR2, or AAV8-sTGFβR2/FGF21 at a dose of 1.5E13 vg/kg each. Wild type mice administered vehicle (WT) were used as control. Means with standard error of the mean are presented.

FIGS. 4A-4C are graphs showing cardiac structure and function ofPKP2cKO mice administered AAV8-sTGFβR2/FGF21 (“sTGFβR2/FGF21”), as measured by left ventricle ejection fraction (LVEF: FIG. 4A), fractional shortening (FS; FIG. 4B) and right ventricle area (RV area: FIG. 4C). Wild type mice administered vehicle (WT) were used as control. Means with standard deviation are presented. * indicates a p value of <0.05 and ** indicates a p value of <0.01 by one-way ANOVA and Dunn's multiple comparisons test.

FIG. 5 is a graph showing arrhythmia burden of PKP2cKO mice administered AAV8-sTGFβR2/FGF21 (“sTGFβR2/FGF21”), measured as the percent of premature ventricular contractions (PVCs) observed in 30 minutes following an isoproterenol challenge. The numbers refer to the number of PVCs observed in 30 minutes following an isoproterenol challenge.

FIGS. 6A and 6B are graphs showing the percentage of collagen in cardiac tissues ofPKP2cKO mice administered AAV8-sTGFβR2/FGF21 (“sTGFβR2/FGF21”), indicative of left ventricle (LV) fibrosis (FIG. 6A), and right ventricle (RV) fibrosis (FIG. 6B). Means with standard deviation are presented.

### DETAILED DESCRIPTION

The present disclosure provides methods of treating an arrhythmogenic cardiomyopathy (ACM) in a subject. The methods generally comprise administering to the subject a gene therapy comprising a first nucleic acid encoding soluble transforming growth factor beta receptor 2 (sTGFβR2) and a second nucleic acid encoding fibroblast growth factor 21 (FGF21).

### Definitions

As used herein, the term “replication-defective adeno-associated virus” refers to an AAV comprising a genome lacking Rep and Cap genes.

As used herein, the term “recombinant AAV genome” or “rAAV genome” refers to a coding sequence operably linked to an exogenous transcriptional regulatory element that mediates expression of the coding sequence when the rAAV genome is introduced into a cell. In certain embodiments, the rAAV genome does not integrate in the chromosomal DNA of the cell. The skilled artisan will appreciate that the portion of a rAAV genome comprising the transcriptional regulatory element operably linked to a coding sequence can be in the sense or antisense orientation relative to direction of transcription of the coding sequence.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may either be wild-type or codon optimized.

As used herein, the term “codon optimized” refers to alteration of a coding sequence of a gene (e.g., by nucleotide substitution) without changing the amino acid sequence of the polypeptide encoded by the coding sequence. Such codon alteration is advantageous in that it may increase the translation efficiency of a coding sequence, and/or prevent recombination with a corresponding sequence of an endogenous gene when a coding sequence is transduced into a cell.

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer sequences. A skilled artisan would appreciate that the promoter and enhancer sequences in a gene may be close in location, and the term “promoter” may refer to a sequence comprising a promoter element and an enhancer sequence. Thus, the term “promoter” does not exclude an enhancer sequence in the sequence. The promoter and enhancer sequences do not need to be derived from the same gene or species, and the sequence of each promoter or enhancer sequence may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer sequences. The coding sequence is “operably linked” to the TRE if the transcription of the coding sequence is controlled or influenced by the TRE. The promoter and enhancer sequences of the TRE may be in any orientation and/or distance from the coding sequence, as long as the desired transcriptional activity is obtained. In certain embodiments, the TRE is upstream from the coding sequence.

As used herein, the term “polyadenylation signal” or “polyadenylation sequence” refers to a DNA sequence that when transcribed into RNA constitutes a polyadenylation signal sequence. The polyadenylation sequence can be native (e.g., with respect to the coding sequence of a gene) or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of a coding sequence of a gene. In certain embodiments, an exogenous polyadenylation sequence can be of the same species (e.g., human), or of a different species (e.g., a virus).

As used herein, the term “effective amount” in the context of the administration of a viral vector (e.g., recombinant AAV) to a subject refers to the amount of the viral vector that achieves a desired prophylactic or therapeutic effect. In the context of the administration of a compound, an effective amount refers to the amount of the compound that achieves a desired prophylactic or therapeutic effect.

As used herein, the term “polynucleotide,” in its broadest sense, includes any compound and/or substance that comprise a polymer of nucleotides linked via a phosphodiester bond.

As used herein, the term “treat,” “treating,” and “treatment” refer to therapeutic or preventative measures described herein. The methods of “treatment” employ administration of a polynucleotide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a non-human mammal. In certain embodiments, the subject is a human. In certain embodiments, the subject is a canine.

### Polynucleotides, Vectors, and Compositions

In one aspect, the methods disclosed herein employ a first nucleic acid encoding soluble transforming growth factor beta receptor 2 (sTGFβR2) and a second nucleic acid encoding fibroblast growth factor 21 (FGF21).

In certain embodiments, the sTGFβR2 coding sequence encodes for a polypeptide comprising all or substantially all of the extracellular portion of TGFβR2 and does not encode for any transmembrane or intracellular aspects of TGFβR2. In certain embodiments, the sTGFβR2 coding sequence encodes for a polypeptide comprising the extracellular portion of wild type TGFβR2. In certain embodiments, the sTGFβR2 coding sequence encodes for a polypeptide comprising the extracellular portion of functional variant of TGFβR2. In certain embodiments, the sTGFβR2 coding sequence encodes a human, mouse, or canine TGFβR2.

In certain embodiments, the sTGFβR2 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 13, 14, or 15. In certain embodiments, the sTGFβR2 coding sequence comprises a nucleotide sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the nucleotide sequence set forth in SEQ ID NO: 1, 2, or 3.

In certain embodiments, the sTGFIPR2 coding sequence encodes for a polypeptide comprising all or substantially all of the extracellular portion of TGFβR2, wherein the extracellular portion of TGFβR2 comprises a secretion signal sequence. In certain embodiments, the secretion signal sequence is an innate secretion signal sequence. In certain embodiments, the secretion signal sequence is a heterologous secretion signal sequence. The heterologous secretion signal sequence can be from the same protein (e.g., TGFβR2) of a different species, or from a different protein of the same or different species. For example, the heterologous secretion signal sequence can be obtained from a TGFβR2 secretion signal sequence of a different species. Exemplary TGFβR2 secretion signal sequences include, without limitation, the secretion signal sequences from human, mouse, and canine TGFβR2. As such, the sTGFj3R2 coding sequence can further encode a heterologous or innate secretion signal sequence. In certain embodiments, the heterologous or innate secretion signal sequence comprises an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the amino acid sequence set forth in SEQ ID NO: 7 or 8. In certain embodiments, the heterologous or innate secretion signal sequence is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 4, 5, or 6.

In certain embodiments, the sTGFβR2 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 16, 17, 18, or 19. In certain embodiments, the sTGFβR2 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 9, 10, 11 or 12.

In certain embodiments, the sTGFβR2 coding sequence further encodes a peptide to achieve half-life extension. Such peptides include, without limitation, an IgG constant domain or fragment thereof (e.g., the Fc domain), human serum albumin (HSA), or albumin-binding polypeptides. In certain embodiments, the sTGFβR2 coding sequence further encodes an Fc domain (referred to herein as a sTGFβR2-Fc coding sequence). Exemplary Fc domains include wild type Fc domains from human, mouse, or canine IgG1, IgG2, IgG3 or IgG4. In certain embodiments, the Fc domain comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 22, 23, or 83. In certain embodiments, the Fc domain is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 20, 21, or 82.

In certain embodiments, the sTGFβR2-Fc coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 26, 27, 73 or 74. In certain embodiments, the sTGFf3R2-Fc coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 24, 25, 71 or 72.

In certain embodiments, the FGF21 coding sequence encodes for a wild type FGF21, or a functional variant thereof. In certain embodiments, the FGF21 coding sequence encodes a human, mouse, or canine FGF21. In certain embodiments, the FGF21 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 32, 33, or 34. In certain embodiments, the FGF21 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 28, 29, 30, or 31.

In certain embodiments, the first nucleic acid encoding soluble transforming growth factor beta receptor 2 (sTGFβR2) is each operably linked to a first transcriptional regulatory element (TRE). In certain embodiments, the second nucleic acid encoding fibroblast growth factor 21 (FGF21) is operably linked to a second TRE. In certain embodiments, the first TRE and the second TRE are the same. In certain embodiments, the first TRE and the second TRE are different. In certain embodiments, the first TRE and the second TRE comprises one or more common elements. The first TRE and the second TRE can be active in any mammalian cells (e.g., human cells, canine cells).

In certain embodiments, the TRE is active in a broad range of mammalian cells. Such TREs may comprise a constitutive promoter and/or enhancer sequences including a cytomegalovirus (CMV) promoter (e.g., comprising a nucleotide sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the nucleotide sequence set forth in SEQ ID NO: 41); a CMV enhancer sequence, a CBA promoter, and the splice acceptor from exon 3 of the rabbit beta-globin gene, collectively called a CAG promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 42); a human calmodulin 1 (CALM1) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 43); a chicken beta actin (CBA) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 44); a CASI promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 45); an smCBA promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 46); a human elongation factor 1 alpha (EFla) promoter (e.g., comprising anucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 35, 36, or 37); an SV40 promoter; a human phosphoglycerate kinase (PGK1) promoter: a human ubiquitin C (Ubc) promoter: a human beta actin promoter; a human neuron-specific enolase (ENO2) promoter; a human beta-glucuronidase (GUSB) promoter; and/or a human Methyl-CpG Binding Protein 2 (MeCP2) promoter. Any of these TREs or elements within these TREs can be combined in any order to drive efficient transcription.

Alternatively, the TRE may be a tissue-specific TRE, i.e., it is active in specific tissue(s) and/or organ(s). A tissue-specific TRE comprises one or more tissue-specific promoter and/or enhancer sequences, and optionally one or more constitutive promoter and/or enhancer sequences. A skilled artisan would appreciate that tissue-specific promoter and/or enhancer sequences can be isolated from genes specifically expressed in the tissue by methods well known in the art.

In certain embodiments, the TRE is liver-specific, i.e., it is active in cells of the liver. Liver-specific TREs include, without limitation, those provided on the Liver Specific Gene Promoter Database (LSPD, rulai.cshl.edu/LSPD/); ahuman alpha-1-antitrypsin (hAAT) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 38); an apolipoprotein E (ApoE) binding site (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 39 or 40); a human albumin (hAlb) or minimal promoter; a transthyretin (TTR) promoter or TTR-minimal promoter (TTRm); an apolipoprotein A1 (APOA1) promoter or minimal promoter; a complement factor B (CFB) promoter: a ketohexokinase (KHK) promoter; a hemopexin (HPX) promoter or minimal promoter: a nicotinamide N-methyltransferase (NNMT) promoter or minimal promoter; a (liver) carboxylesterase 1 (CES1) promoter or minimal promoter; a protein C (PROC) promoter or minimal promoter; an apolipoprotein C3 (APOC3) promoter or minimal promoter: a mannan-binding lectin serine protease 2 (MASP2) promoter or minimal promoter; a hepcidin antimicrobial peptide (HAMP) promoter or minimal promoter; and a serpin peptidase inhibitor, clade C (antithrombin), member 1 (SERPINCI) promoter or minimal promoter.

The TRE may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, without limitation, a metallothionine promoter, a glucocorticoid promoter, a progesterone promoter, and a tetracycline promoter.

In certain embodiments, the first nucleic acid and/or the second nucleic acid comprises two or more TREs, optionally comprising at least one of the TREs disclosed herein. A skilled person in the art would appreciate that any of these TREs can be combined in any order, and combinations of a constitutive TRE and a tissue-specific TRE can drive efficient and tissue-specific transcription.

In certain embodiments, the TRE can further comprise an intron sequence. Such introns can increase transgene expression, for example, by reducing transcriptional silencing and enhancing mRNA export from the nucleus to the cytoplasm. The intron can comprise a native intron sequence of sTGFβR2 or FGF21, an intron sequence from the same genes of a different species, an intron sequence from a different gene from the same species, and/or a synthetic intron sequence. A skilled worker will appreciate that synthetic intron sequences can be designed to mediate RNA splicing by introducing any consensus splicing motifs known in the art (e.g., in Sibley et al. Nature Reviews Genetics. 2016, 17:407-21, which is incorporated by reference herein in its entirety). Exemplary intron sequences are provided in Lu et al., Molecular Therapy. 2013, 21(5): 954-63, and Lu et al., Hum. Gene Ther. 2017, 28(1): 125-34, which are incorporated by reference herein in their entirety. Suitable intron sequence include, without limitation, a minute virus of mouse (MVM) intron (e.g., comprising a nucleotide sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the nucleotide sequence set forth in SEQ ID NO: 47); a β-globin intron sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 48); and an SV40 intron sequence.

In certain embodiments, the first nucleic acid and/or the second nucleic acid further comprises a post-transcriptional regulatory element. The post-transcriptional regulatory element may be any sequence that effectively terminates transcription, and a skilled artisan would appreciate that such sequences can be isolated from any genes that are expressed in the cell in which transcription of the coding sequence is desired.

In certain embodiments, the post-transcriptional regulatory element comprises a polyadenylation signal sequence. In certain embodiments, the polyadenylation signal sequence is identical or substantially identical to the endogenous polyadenylation sequence of the sTGFβR2 or FGF21 gene. In certain embodiments, the polyadenylation signal sequence is an exogenous polyadenylation signal sequence. In certain embodiments, the polyadenylation signal sequence is an SV40 polvadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 51); a bovine growth hormone polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 52); a rabbit beta globin polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 53); or a human growth hormone polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 54).

In certain embodiments, the post-transcriptional regulatory element comprises a Woodchuck Hepatitis Virus (WHV) post-transcriptional regulatory element (WPRE). In certain embodiments, the post-transcriptional regulatory element comprises a WPRE sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 49); or a WPRE3 sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50 or 70).

The first nucleic acid and/or the second nucleic acid described herein can be transcribed from an expression vector (e.g., a recombinant expression vector). In certain embodiments, the first nucleic acid is comprised within a first vector, and the second nucleic acid is comprised within a second vector. In certain embodiments, the first nucleic acid and the second nucleic acid are comprised within a single vector (e.g., a bigenic vector). Where the first nucleic acid and the second nucleic acid are comprised within a single vector, the first nucleic acid and the second nucleic acid may be separated by a polycistronic element.

In certain embodiments, the polycistronic element comprises anucleotide sequence that encodes for an internal ribosome entry site (IRES). An IRES is an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). In certain embodiments, the polycistronic element comprises a nucleotide sequence that encodes for a 2A sequence. A 2A sequence refers to an oligopeptide that allow multiple proteins to be encoded as polyproteins, which dissociate into component proteins upon translation. Various 2A sequences are known to those of skill in the art, including, without limitation, those found in members of the Picomaviridae virus family, e.g., foot-and-mouth disease virus (FMDV), equine rhinitis A virus (ERAVO), Thosea asigna virus (TaV), and porcine tescho virus-1 (PTV-1); and carioviruses such as Theilovirus and encephalomyocarditis viruses. 2A sequences derived from FMDV, ERAV, PTV-1, and TaV are referred to herein as “F2A,” “E2A,” “P2A,” and “T2A,” respectively. In certain embodiments, the polycistronic element comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 55, 56, or 57.

In certain embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In certain embodiments, the non-viral vector is a DNA plasmid vector. In certain embodiments, the non-viral vector is a transposon-based vector. In certain embodiments, the non-viral vector is a PiggyBac-based vector, or a Sleeping Beauty-based vector.

In certain embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV1, AAV2. AAV3. AAV4, AAV5, AAV6. AAV7, AAV8, AAV9), retrovirus vectors (e.g., MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector. Newcastle disease virus vectors, poxvirus vectors, and picornavirus vectors (e.g., Coxsackievirus). In certain embodiments, the viral vector is selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

In certain embodiments, the vector is an AAV vector. In certain embodiments, the vector is a single-stranded AAV. In certain embodiments, the vector is a self-complementary AAV.

In certain embodiments, the vector is an AAV vector comprised within a recombinant AAV (rAAV). In certain embodiments, the rAAV comprises an AAV capsid comprising an AAV capsid protein, and a rAAV genome.

A capsid protein from any capsid known the art can be used in the rAAV compositions disclosed herein, including, without limitation, a capsid protein from an AAV1, AAV2, AAV3. AAV4. AAV5. AAV6. AAV7. AAV8, or AAV9 serotype. The capsid protein can be from a clade A, clade B, clade C, clade D, clade E, clade F, clade G, clade H, clade I, AAVgo.1. AAV3, AAV4, AAV10, AAV11, AAV12, rh.32, rh32.33, rh.33, rh.34, BAAV, or AAV5 capsid protein, or an engineered variant thereof. In certain embodiments, the capsid protein is from AAV8. In certain embodiments, the capsid protein is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 62. In certain embodiments, the capsid protein comprises an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the amino acid sequence of amino acids 1-738 of SEQ ID NO: 63; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 138-738 of SEQ ID NO: 63; and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 204-738 of SEQ ID NO: 63. In certain embodiments, the capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 63; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 64; and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 65.

In certain embodiments, the rAAV comprises an AAV capsid comprising an AAV capsid protein, and a rAAV genome comprising a nucleic acid encoding sTGFβR2. In certain embodiments, the rAAV comprises an AAV capsid comprising an AAV capsid protein, and a rAAV genome comprising a nucleic acid encoding sTGFβR2-Fc. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 26. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 66.

In certain embodiments, the rAAV comprises an AAV capsid comprising an AAV capsid protein, and a rAAV genome comprising a nucleic acid encoding sTGFβR2. In certain embodiments, the rAAV comprises an AAV capsid comprising an AAV capsid protein, and a rAAV genome comprising a nucleic acid encoding sTGFβR2-Fc. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 73. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 78.

In certain embodiments, the rAAV comprises an AAV capsid comprising an AAV capsid protein, and a rAAV genome comprising a nucleic acid encoding FGF21. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 33. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 67.

In certain embodiments, the rAAV comprises an AAV capsid comprising an AAV capsid protein, and a rAAV genome comprising a nucleic acid encoding FGF21. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 34. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 80.

In certain embodiments, the rAAV comprises an AAV capsid comprising an AAV capsid protein, and a rAAV genome comprising a nucleic acid encoding sTGFβR2 and a nucleic acid encoding FGF21. In certain embodiments, the rAAV comprises an AAV capsid comprising an AAV capsid protein, and a rAAV genome comprising a nucleic acid encoding sTGFβR2-Fc and anucleic acid encoding FGF21. In certain embodiments, the nucleic acid encoding sTGFβR2 or sTGFβR2-Fc and the nucleic acid encoding FGF21 may be separated by a polycistronic element.

In certain embodiments, the rAAV genome further comprises a 5′ inverted terminal repeat (5′ ITR) nucleotide sequence, and a 3′ inverted terminal repeat (3′ ITR) nucleotide sequence 3′ of the coding sequence. In certain embodiments, the rAAV genome comprises a 5′ ITR 5′ of the TRE, and a 3′ ITR 3′ of the coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NO: 58, 59, 60, 61, 76, and 77.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 58, 59, or 76. In certain embodiments, the 3′ ITR nucleotide sequence has at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the nucleotide sequence set forth in SEQ ID NO: 60, 61, or 77. In certain embodiments, the 5′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59, and the 3′ ITR nucleotide sequence has at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the nucleotide sequence set forth in SEQ ID NO: 61. In certain embodiments, the 5′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 76, and the 3′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 77.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59); a transcriptional regulatory element operably linked to a nucleic acid encoding sTGFβR2 or sTGFβR2-Fc (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 2, 11, 20, 24, 25, 38, 39, 40, 47, and/or 48); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, or 75); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 67.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 76); a transcriptional regulatory element operably linked to a nucleic acid encoding sTGFβR2 or sTGFβR2-Fc (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 3, 12, 35, 36, 71, and/or 72); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, or 75); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 77). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 79.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59); a transcriptional regulatory element operably linked to a nucleic acid encoding FGF21 (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 30, 38, 39, 40, 47, and/or 48); a post-transcriptional regulatory element (e.g., comprising a nucleotide sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, 75); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 69.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to the nucleotide sequence set forth in SEQ ID NO: 76); a transcriptional regulatory element operably linked to a nucleic acid encoding FGF21 (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 31, 35, and/or 36); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, or 75); and a 3 ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 77). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 81.

In certain embodiments, the rAAV comprises: (a) an AAV capsid protein comprising the amino acid sequence of amino acids 1-738 of SEQ ID NO: 63, the amino acid sequence of amino acids 138-738 of SEQ ID NO: 63, and/or the amino acid sequence of amino acids 204-738 of SEQ ID NO: 63; and (b) an rAAV genome comprising the nucleotide sequence set forth in any one of SEQ ID NOs: 66, 67, 68, or 69. In certain embodiments, the rAAV comprises (a) an AAV capsid protein comprising the amino acid sequence of amino acids 1-738 of SEQ ID NO: 63, and an rAAV genome comprising the nucleotide sequence set forth in any one of SEQ ID NOs: 66, 67, 68, or 69; (b) an AAV capsid protein comprising the amino acid sequence of amino acids 138-738 of SEQ ID NO: 63, and an rAAV genome comprising the nucleotide sequence set forth in any one of SEQ ID NOs: 66, 67, 68, or 69; and/or (c) an AAV capsid protein comprising the amino acid sequence of amino acids 204-738 of SEQ ID NO: 6³, and an rAAV genome comprising the nucleotide sequence set forth in any one of SEQ ID NOs: 66, 67, 68, or 69;

In certain embodiments, the rAAV comprises: (a) an AAV capsid protein comprising the amino acid sequence of amino acids 1-738 of SEQ ID NO: 63, the amino acid sequence of amino acids 138-738 of SEQ ID NO: 63, and/or the amino acid sequence of amino acids 204-738 of SEQ ID NO: 63; and (b) an rAAV genome comprising the nucleotide sequence set forth in any one of SEQ ID NOs: 66, 67, 68, or 69. In certain embodiments, the rAAV comprises (a) an AAV capsid protein comprising the amino acid sequence of amino acids 1-738 of SEQ ID NO: 63, and an rAAV genome comprising the nucleotide sequence set forth in any one of SEQ ID NOs: 78, 79, 80, or 81; (b) an AAV capsid protein comprising the amino acid sequence of amino acids 138-738 of SEQ ID NO: 63, and an rAAV genome comprising the nucleotide sequence set forth in any one of SEQ ID NOs: 66, 67, 68, or 69; and/or (c) an AAV capsid protein comprising the amino acid sequence of amino acids 204-738 of SEQ ID NO: 6³, and an rAAV genome comprising the nucleotide sequence set forth in any one of SEQ ID NOs: 78, 79, 80, or 81;

In another aspect, the present disclosure provides a polynucleotide comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 66, 67, 68, or 69.

In another aspect, the present disclosure provides a polynucleotide comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 78, 79, 80, or 81.

The polynucleotide can comprise DNA, RNA, modified DNA, modified RNA, or a combination thereof. In certain embodiments, the polynucleotide is an expression vector. In certain embodiments, the polynucleotide is comprised within a viral vector. In certain embodiments, the polynucleotide is comprised within a plasmid vector.

In another aspect, the present disclosure provides pharmaceutical compositions comprising a rAAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy,” 20th edition, Lippincott, Williams. & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

### Methods of Treatment

Arrhythmogenic cardiomyopathy (ACM) is an inherited heart muscle disorder characterized by redistribution of junctional proteins, arrhythmias, and progressive myocardial injury. Pathological features of ACM include loss of myocytes and fibrofatty replacement of right ventricular myocardium. Biventricular involvement is often observed. ACM is a cell junction cardiomyopathy, typically caused by genetic abnormalities of cardiac desmosomes, which results in the detachment of myocytes and affects intracellular signal transduction.

ACM is a genetically heterogeneous disorder, for which there are several genes in which mutations are known to be causative. Genes in which mutations have been reported to cause ACM include: junction plakoglobin (JUP): desmoplakin (DSP); plakophilin-2 (PKP2); desmoglein-2 (DSG2); desmocollin-2 (DSC2); transmembrane protein 43 (TMEM43); lamin A/C (LMNA); desmin (DES); alpha-T-catenin (CTNNA3): phospholamban (PLN); transforming growth factor 3 (TGFB3); titin (TTN); sodium voltage-gated channel alpha subunit 5 (SCN5A; Na,1.5); and cadherin C (CDH2). Such genes have been studied and have progressed understanding of the genetic underpinnings of ACM. However, there remain cases of ACM that are genetically elusive.

While the original ACM disease phenotype was characterized by predominant right ventricle (RV) involvement (ARVC), with minor and late left ventricle (LV) disease, clinical variants characterized by early and greater LV involvement, which may parallel (e.g., biventricular ACM) or exceed (e.g., left-dominant ACM, or ALVC) the severity of RV involvement, have been increasingly reported.

There is not a single gold standard method for the diagnosis of ACM, and as such, the best strategy consists of combining multiple sources of clinical information, such as genetic, electrocardiographic, arrhythmic, morpho-functional, and histopathological findings. Current international criteria for ARVC and ALVC diagnosis include assessing morpho-functional ventricular abnormalities (i.e., global and regional ventricular dilatation and systolic dysfunction), structural myocardial abnormalities (i.e., for existence of fibrous or fibro-fatty myocardial replacement), echocardiographic abnormalities (e.g., depolarization and repolarization abnormalities), ventricular arrhythmias, and/or family history and molecular genetics. Phenotypic variants of ACM such as ARVC, biventricular ACM, and ALVC, are diagnosed based on a scoring system for fulfilling the above-mentioned criteria.

In another aspect, the present disclosure provides methods for treating an arrhythmogenic cardiomyopathy (ACM) in a subject. The methods generally comprise administering to the subject an effective amount of a gene therapy comprising a first nucleic acid encoding soluble transforming growth factor beta receptor 2 (sTGFβR2) and a second nucleic acid encoding fibroblast growth factor 21 (FGF21).

In certain embodiments, a method for treating an ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating an ACM in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating an ACM in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating an ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating an ACM in a subject comprises administering: (a) a first rAAV comprising an AAV capsid comprising an AAV capsid protein, and a first rAAV genome comprising a nucleic acid encoding the amino acid sequence set forth in SEQ ID NO: 73; and (b) a second rAAV comprising an AAV capsid comprising an AAV capsid protein, and a second rAAV genome comprising a nucleic acid encoding the amino acid sequence set forth in SEQ ID NO: 74. In certain embodiments, a method for treating an ACM in a subject comprises administering: (a) a first rAAV comprising an AAV capsid comprising an AAV capsid protein, and a first rAAV genome comprising the nucleotide sequence set forth in SEQ ID NO: 78 or 79; and (b) a second rAAV comprising an AAV capsid comprising an AAV capsid protein, and a second rAAV genome comprising the nucleotide sequence set forth in SEQ ID NO: 80 or 81.

In certain embodiments, a method for treating an ACM in a subject comprises administering an rAAV comprising an AAV capsid comprising an AAV capsid protein, and an rAAV genome comprising: a first nucleic acid encoding sTGFβR2 and a second nucleic acid encoding FGF21.

In certain embodiments, a method for treating an ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating an ACM in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating an ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating an ACM in a subject comprises administering an rAAV comprising an AAV capsid comprising an AAV capsid protein, and an rAAV genome comprising: a first nucleic acid encoding the amino acid sequence set forth in SEQ ID NO: 73 and a second nucleic acid encoding the amino acid sequence set forth in SEQ ID NO: 34, wherein the first nucleic acid and the second nucleic acid are separated by a polycistronic element.

In certain embodiments, a method for treating an ACM in a subject further comprises administering to the subject an effective amount of one or more additional therapeutics to treat an ACM (e.g., a non-gene therapy therapeutic to treat an ACM). Such additional therapeutics to treat an ACM may include, without limitation, angiotensin-converting enzyme (ACE) inhibitors, angiotensin II receptor blockers, anti-arrhythmia agents, beta blockers, blood thinners, and diuretics.

In certain embodiments, a method for treating an ACM in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of one or more additional therapeutics to treat an ACM. In certain embodiments, a method for treating an ACM in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of one or more additional therapeutics to treat an ACM. In certain embodiments, a method for treating an ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21; and (b) an effective amount of one or more additional therapeutics to treat an ACM. In certain embodiments, a method for treating an ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element; and (b) an effective amount of one or more additional therapeutics to treat an ACM.

In certain embodiments, the one or more additional therapeutics to treat an ACM is administered at the same time as the gene therapy. In certain embodiments, the one or more additional therapeutics to treat an ACM is administered at a different time as the gene therapy.

In certain embodiments, a method of treating an ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21). In certain embodiments, a method of treating an ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21).

In certain embodiments, the ACM is selected from the group consisting of arrhythmogenic right ventricular cardiomyopathy (ARVC), arrhythmogenic left ventricular cardiomyopathy (ALVC), and biventricular arrhythmogenic cardiomyopathy. In certain embodiments, the ACM is ARVC.

In certain embodiments, the ACM is ACM associated with a loss of function in the gene encoding plakophilin-2 (PKP2). In certain embodiments, the ACM is a PKP2 mutation-associated ACM. In certain embodiments, the ACM is ACM associated with a loss of function in the gene encoding desmoglein-2 (DSG2). In certain embodiments, the ACM is a DSG2 mutation-associated ACM. In certain embodiments, the ACM is ACM associated with a loss of function in the gene encoding desmoplakin (DSP). In certain embodiments, the ACM is a DSP mutation-associated ACM. In certain embodiments, the ACM is ACM associated with a loss of function in a gene encoding a desmocollin (DSC), optionally in the gene encoding desmocollin-2 (DSC2). In certain embodiments, the ACM is a DSC mutation-associated ACM. In certain embodiments, the ACM is a DSC2 mutation-associated ACM.

In certain embodiments, the ACM is ACM associated with a loss of function in one or more genes selected from the group consisting of PKP2, DSG2, DSP, and a DSC gene. In certain embodiments, the ACM is ACM associated with a loss of function in one or more genes selected from the group consisting of PKP2, DSG2, DSP, and DSC2. In certain embodiments, the ACM is a PKP2 mutation-associated ACM, a DSG2 mutation-associated ACM, a DSP mutation-associated ACM, a DSC mutation-associated ACM, and/or a DSC2 mutation-associated ACM.

The PKP2 gene is the most common gene associated with ACM, and encodes plakophilin-2 (PKP2), a component of the desmosome critical for cell-cell adhesion. In certain embodiments, the ACM is PKP2 mutation-associated ACM. In certain embodiments, a method for treating PKP2 mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating PKP2 mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating PKP2 mutation-associated ACM in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating PKP2 mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating PKP2 mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 73; and (b) a second rAAV comprising an AAV capsid comprising an AAV capsid protein, and a second rAAV genome comprising a nucleic acid encoding the amino acid sequence set forth in SEQ ID NO: 74. In certain embodiments, a method for treating PKP2 mutation-associated ACM in a subject comprises administering: (a) a first rAAV comprising an AAV capsid comprising an AAV capsid protein, and a first rAAV genome comprising the nucleotide sequence set forth in SEQ ID NO: 78 or 79; and (b) a second rAAV comprising an AAV capsid comprising an AAV capsid protein, and a second rAAV genome comprising the nucleotide sequence set forth in SEQ ID NO: 80 or 81.

In certain embodiments, a method for treating PKP2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21.

In certain embodiments, a method for treating PKP2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating PKP2 mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating PKP2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating PKP2 mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 73 and a second nucleic acid encoding the amino acid sequence set forth in SEQ ID NO: 34, wherein the first nucleic acid and the second nucleic acid are separated by a polycistronic element.

In certain embodiments, a method for treating PKP2 mutation-associated ACM in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of one or more additional therapeutics to treat an ACM. In certain embodiments, a method for treating PKP2 mutation-associated ACM in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of one or more additional therapeutics to treat an ACM. In certain embodiments, a method for treating PKP2 mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21; and (b) an effective amount of one or more additional therapeutics to treat an ACM. In certain embodiments, a method for treating PKP2 mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element; and (b) an effective amount of one or more additional therapeutics to treat an ACM.

In certain embodiments, a method of treating PKP2 mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21). In certain embodiments, a method of treating PKP2 mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21).

It is estimated that 5-10% of cases with ACM carry a pathogenic mutation in the DSG2 gene, which encodes desmoglein-2, a transmembrane glycoprotein that mediates the calcium-dependent cell-cell adhesion between cardiomyocytes. In certain embodiments, the ACM is DSG2 mutation-associated ACM. In certain embodiments, a method for treating DSG2 mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating DSG2 mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating DSG2 mutation-associated ACM in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating DSG2 mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating DSG2 mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 73; and (b) a second rAAV comprising an AAV capsid comprising an AAV capsid protein, and a second rAAV genome comprising a nucleic acid encoding the amino acid sequence set forth in SEQ ID NO: 74. In certain embodiments, a method for treating DSG2 mutation-associated ACM in a subject comprises administering: (a) a first rAAV comprising an AAV capsid comprising an AAV capsid protein, and a first rAAV genome comprising the nucleotide sequence set forth in SEQ ID NO: 78 or 79; and (b) a second rAAV comprising an AAV capsid comprising an AAV capsid protein, and a second rAAV genome comprising the nucleotide sequence set forth in SEQ ID NO: 80 or 81.

In certain embodiments, a method for treating DSG2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21.

In certain embodiments, a method for treating DSG2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating DSG2 mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating DSG2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating DSG2 mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 73 and a second nucleic acid encoding the amino acid sequence set forth in SEQ ID NO: 34, wherein the first nucleic acid and the second nucleic acid are separated by a polycistronic element.

In certain embodiments, a method for treating DSG2 mutation-associated ACM in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of one or more additional therapeutics to treat an ACM. In certain embodiments, a method for treating DSG2 mutation-associated ACM in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of one or more additional therapeutics to treat an ACM. In certain embodiments, a method for treating DSG2 mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21; and (b) an effective amount of one or more additional therapeutics to treat an ACM. In certain embodiments, a method for treating DSG2 mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element; and (b) an effective amount of one or more additional therapeutics to treat an ACM.

In certain embodiments, a method of treating DSG2 mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21). In certain embodiments, a method of treating DSG2 mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21).

Variants in the DSP gene, which encodes desmoplakin, the most prevalent component of desmosome, have been associated with ACM phenotypes. Variants causing a DSP loss of function lead to the failure of intercellular adhesion and have been associated with histological findings of inflammatory infiltrates and fibrosis of the left ventricular myocardium. In certain embodiments, the ACM is DSP mutation-associated ACM. In certain embodiments, a method for treating DSP mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating DSP mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating DSP mutation-associated ACM in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating DSP mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating DSP mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 73; and (b) a second rAAV comprising an AAV capsid comprising an AAV capsid protein, and a second rAAV genome comprising a nucleic acid encoding the amino acid sequence set forth in SEQ ID NO: 74. In certain embodiments, a method for treating DSP mutation-associated ACM in a subject comprises administering: (a) a first rAAV comprising an AAV capsid comprising an AAV capsid protein, and a first rAAV genome comprising the nucleotide sequence set forth in SEQ ID NO: 78 or 79; and (b) a second rAAV comprising an AAV capsid comprising an AAV capsid protein, and a second rAAV genome comprising the nucleotide sequence set forth in SEQ ID NO: 80 or 81.

In certain embodiments, a method for treating DSP mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21.

In certain embodiments, a method for treating DSP mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating DSP mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating DSP mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating DSP mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 73 and a second nucleic acid encoding the amino acid sequence set forth in SEQ ID NO: 34, wherein the first nucleic acid and the second nucleic acid are separated by a polycistronic element.

In certain embodiments, a method for treating DSP mutation-associated ACM in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of one or more additional therapeutics to treat an ACM. In certain embodiments, a method for treating DSP mutation-associated ACM in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of one or more additional therapeutics to treat an ACM. In certain embodiments, a method for treating DSP mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21; and (b) an effective amount of one or more additional therapeutics to treat an ACM. In certain embodiments, a method for treating DSP mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element; and (b) an effective amount of one or more additional therapeutics to treat an ACM.

In certain embodiments, a method of treating DSP mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21). In certain embodiments, a method of treating DSP mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21).

Desmocollins are desmosomal cadherins, type I transmembrane glycoproteins that mediate cell-cell interactions. Variants in desmocollin-2 (DSC2), especially homozygous truncating variants, have been found to be causative for ACM. In certain embodiments, the ACM is a DSC mutation-associated ACM, e.g., a DSC2 mutation-associated ACM. In certain embodiments, a method for treating DSC2 mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating DSC2 mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating DSC2 mutation-associated ACM in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating DSC2 mutation-associated ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating DSC2 mutation-associated ACM in a subject comprises administering: (a) a first rAAV comprising an AAV capsid comprising an AAV capsid protein, and a first rAAV genome comprising a nucleic acid encoding the amino acid sequence set forth in SEQ ID NO: 73; and (b) a second rAAV comprising an AAV capsid comprising an AAV capsid protein, and a second rAAV genome comprising a nucleic acid encoding the amino acid sequence set forth in SEQ ID NO: 74. In certain embodiments, a method for treating DSC2 mutation-associated ACM in a subject comprises administering: (a) a first rAAV comprising an AAV capsid comprising an AAV capsid protein, and a first rAAV genome comprising the nucleotide sequence set forth in SEQ ID NO: 78 or 79; and (b) a second rAAV comprising an AAV capsid comprising an AAV capsid protein, and a second rAAV genome comprising the nucleotide sequence set forth in SEQ ID NO: 80 or 81.

In certain embodiments, a method for treating DSC2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21.

In certain embodiments, a method for treating DSC2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating DSC2 mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating DSC2 mutation-associated ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating DSC2 mutation-associated ACM in a subject comprises administering an rAAV comprising an AAV capsid comprising an AAV capsid protein, and an rAAV genome comprising: a first nucleic acid encoding the amino acid sequence set forth in SEQ ID NO: 73 and a second nucleic acid encoding the amino acid sequence set forth in SEQ ID NO: 34, wherein the first nucleic acid and the second nucleic acid are separated by a polycistronic element.

In certain embodiments, a method for treating DSC2 mutation-associated ACM in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of one or more additional therapeutics to treat an ACM. In certain embodiments, a method for treating DSC2 mutation-associated ACM in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of one or more additional therapeutics to treat an ACM. In certain embodiments, a method for treating DSC2 mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21; and (b) an effective amount of one or more additional therapeutics to treat an ACM. In certain embodiments, a method for treating DSC2 mutation-associated ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element; and (b) an effective amount of one or more additional therapeutics to treat an ACM.

In certain embodiments, a method of treating DSC2 mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21). In certain embodiments, a method of treating DSC2 mutation-associated ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21).

Certain cases of ACM remain genetically elusive. In certain embodiments, the ACM genetically elusive, i.e., ACM with no known genetic cause. In certain embodiments, a method for treating a genetically elusive ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating a genetically elusive ACM in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating a genetically elusive ACM in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating a genetically elusive ACM in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating a genetically elusive ACM in a subject comprises administering: (a) a first rAAV comprising an AAV capsid comprising an AAV capsid protein, and a first rAAV genome comprising a nucleic acid encoding the amino acid sequence set forth in SEQ ID NO: 73; and (b) a second rAAV comprising an AAV capsid comprising an AAV capsid protein, and a second rAAV genome comprising a nucleic acid encoding the amino acid sequence set forth in SEQ ID NO: 74. In certain embodiments, a method for treating a genetically elusive ACM in a subject comprises administering: (a) a first rAAV comprising an AAV capsid comprising an AAV capsid protein, and a first rAAV genome comprising the nucleotide sequence set forth in SEQ ID NO: 78 or 79; and (b) a second rAAV comprising an AAV capsid comprising an AAV capsid protein, and a second rAAV genome comprising the nucleotide sequence set forth in SEQ ID NO: 80 or 81.

In certain embodiments, a method for treating a genetically elusive ACM in a subject comprises administering an rAAV comprising an AAV capsid comprising an AAV capsid protein, and an rAAV genome comprising: a first nucleic acid encoding sTGFβR2 and a second nucleic acid encoding FGF21.

In certain embodiments, a method for treating a genetically elusive ACM in a subject comprises administering an rAAV comprising an AAV capsid comprising an AAV capsid protein, and an rAAV genome comprising: a first nucleic acid encoding sTGFj3R2 and a second nucleic acid encoding FGF21, wherein the first nucleic acid and the second nucleic acid are separated by a polycistronic element. In certain embodiments, a method for treating a genetically elusive ACM in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating a genetically elusive ACM in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating a genetically elusive ACM in a subject comprises administering an rAAV comprising an AAV capsid comprising an AAV capsid protein, and an rAAV genome comprising: a first nucleic acid encoding the amino acid sequence set forth in SEQ ID NO: 73 and a second nucleic acid encoding the amino acid sequence set forth in SEQ ID NO: 34, wherein the first nucleic acid and the second nucleic acid are separated by a polycistronic element.

In certain embodiments, a method for treating a genetically elusive ACM in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of one or more additional therapeutics to treat an ACM. In certain embodiments, a method for treating a genetically elusive ACM in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of one or more additional therapeutics to treat an ACM. In certain embodiments, a method for treating a genetically elusive ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21; and (b) an effective amount of one or more additional therapeutics to treat an ACM. In certain embodiments, a method for treating a genetically elusive ACM in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element; and (b) an effective amount of one or more additional therapeutics to treat an ACM.

In certain embodiments, a method of treating a genetically elusive ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFfPR2) and a second nucleic acid encoding fibroblast growth factor 21 (FGF21). In certain embodiments, a method of treating a genetically elusive ACM in a subject comprises administering to a subject that has received an effective amount of a non-gene therapy therapeutic to treat an ACM, an effective amount of a gene therapy comprising a first nucleic acid encoding soluble transforming growth factor beta receptor 2 (sTGFβR2) and a second nucleic acid encoding fibroblast growth factor 21 (FGF21).

Ejection fraction refers to how well a heart pumps blood and is calculated by measuring the amount of blood pumped out of a heart's ventricles each time it contracts. LVEF refers to how much blood is pumped out of the left ventricle to most of the body's organs with each contraction. LVEF helps determine the severity of dysfunction on the left side of the heart. Fractional shortening (FS) refers to the reduction of the length of the end-diastolic diameter that occurs by the end of systole. Both ejection fraction and FS are measures of the heart's muscular contractility and health. In certain embodiments, a method of treating an ACM in a subject further comprises determining a base level of fractional shortening (FS) and/or left ventricular ejection fraction (LVEF) in the subject prior to administration of the gene therapy. In certain embodiments, the gene therapy results in a reduction in the level of FS and/or LVEF in the subject at a duration after administration of the gene therapy, as compared to the level of FS and/or LVEF in the subject. The duration in which a subject exhibits a reduction in the level of FS and/or LVEF compared to the subject's base level of FS and/or LVEF, can be 1 week, 2 weeks, 3 weeks, 4 weeks, 1 month, 2 months, 3 months, 4 months, 5 months, 6 months, 7 months. 8 months, 9 months, 10 months, 11 months, 1 year, 2 years, 3 years, 4 years, 5 years, 6 years, 7 years, 8 years, 9 years, 10 years, or longer.

It is known in the art that complex cross-talk exists between TGFβ and FGFR signaling pathways. It is also known in the art that increased Nuclear Factor Kappa B (NF-κB) activation is associated with heart arrhythmia (e.g., atrial fibrillation). While not being bound by theory, it is believed that a gene therapy comprising a first nucleic acid encoding soluble transforming growth factor beta receptor (sTGFβR2) and a second nucleic acid encoding fibroblast growth factor 21 (FGF21) can down regulate NF-κB activation, resulting in the treatment of heart arrhythmia (i.e., a reduction in the degree or severity of arrhythmia).

In certain embodiments, the subject having an ACM has arrhythmia. In certain embodiments, a method of treating an ACM in a subject further comprises determining a base level of arrhythmia in the subject prior to administration of the gene therapy. In certain embodiments, a method of treating an ACM in a subject further comprises determining a base level of NF-κB activation in the subject prior to administration of the gene therapy. In certain embodiments, the treatment results in a reduction in arrhythmia and/or NF-κB activation in the subject at a duration after administration of the gene therapy, as compared to the degree of arrhythmia and/or NF-κB in the subject prior to administration of the gene therapy. The duration in which a subject exhibits a reduction in the degree of arrhythmia and/or NF-κB activation compared to the subject's base level of arrhythmia and/or NF-κB activation, can be 1 week, 2 weeks, 3 weeks, 4 weeks, 1 month, 2 months, 3 months, 4 months, 5 months, 6 months. 7 months, 8 months, 9 months, 10 months. 11 months, I year. 2 years, 3 years. 4 years, 5 years. 6 years, 7 years, 8 years, 9 years, 10 years, or longer.

In certain embodiments, the gene therapy is administered to the subject intravenously, intraperitoneally, subcutaneously, intramuscularly, intrathecally, or intradermally.

In certain embodiments, the subject is a member of any mammalian or non-mammalian species. Suitable subjects include, without limitation, humans, non-human primates, canines, felines, ungulates (e.g., equine, bovine, swine (e.g., pig)), avians, rodents (e.g., rats, mice), and other subjects. In certain embodiments, the subject is human. In certain embodiments, the subject is canine.

### EXAMPLES

The following examples are offered by way of illustration, and not by way of limitation.

### Example 1: sTGFβR2 and FGF21 Recombinant AAV Vectors

This example provides sTGFβR2 and FGF21 recombinant adeno-associated virus (rAAV) vectors for expression of sTGFβR2 and FGF21 in a cell (e.g., a liver cell) to which the vectors are transduced.

rAAV-sTGFβR2 comprises a rAAV genome comprising from 5′ to 3′ the following genetic elements: a 5′ ITR element, an EF-1α promoter, a sTGFβR2-Fc coding sequence, a WPRE3 sequence, an SV40 polyadenylation signal, and a 3′ ITR element. The sequences of these elements are set forth in Table 1. This vector is capable of expressing a sTGFβR2-Fc fusion protein in a cell (e.g., a liver cell) to which the vector is transduced.

rAAV-FGF21 comprises a rAAV genome comprising from 5′ to 3′ the following genetic elements: a 5′ ITR element, an EF-1α promoter, a FGF21 coding sequence, a WPRE3 sequence, an SV40 polyadenylation signal, and a 3′ ITR element. The sequences of these elements are set forth in Table 1. This vector is capable of expressing a FGF21 protein in a cell (e.g., a liver cell) to which the vector is transduced.

TABLE 1
Genetic Elements in rAAV-sTGFβR2 and rAAV-FGF21

| | rAAV-sTGFβR2 | rAAV-FGF21 |
|---|---|---|
| Genetic Element | SEQ ID NO: | |
| 5′ ITR element | 76 | 76 |
| EF-1α promoter sequence | 35 | 36 |
| Coding sequence | 71 | 31 |
| Encoded sequence | 73 | 34 |
| WPRE3 sequence | 50 | 50 |
| SV40 polyadenylation signal | 75 | 75 |
| 3′ ITR element | 77 | 77 |
| rAAV genome (promoter to | 78 | 80 |
| polyadenylation signal) | | |
| rAAV genome | 79 | 81 |
| (5′ ITR to 3′ ITR) | | |

The rAAV vectors disclosed herein can be packaged in an AAV capsid, such as, without limitation, an AAV8 capsid. In general, viral particles were generated using standard triple transfection of HEK293T cells and affinity and anion purification. See, e.g., Davidsohn et al (2019) Proc. Natl. Acad. Sci. 116(47): 23505-23511, and Nass et al. (2018) Mol. Ther. Methods Clin. Dev. 9: 33-46, The packaged viral particles can be administered to a wild-type animal, or a subject suffering from arrhythmogenic right ventricular cardiomyopathy (ARVC).

### Example 2: Expression of sTGFβR2 and FGF21 in Mouse Model of ARVC

Dsg2^{mut/mut} mice were generated as described in Chelko et al. (2016) JCI Insight 1(5): e85923. Dsg2^{mut/mut} mice contain a loss of exons 4 and 5 of Dsg2 causing a frameshift mutation and premature truncation, which is predicted to be degraded by nonsense-mediated mRNA decay. Dsg2^{mut/mut} mice have previously been shown to develop cardiomyopathy by 8 weeks of age compared with wild-type littermates, and to have significant reduction in heart function.

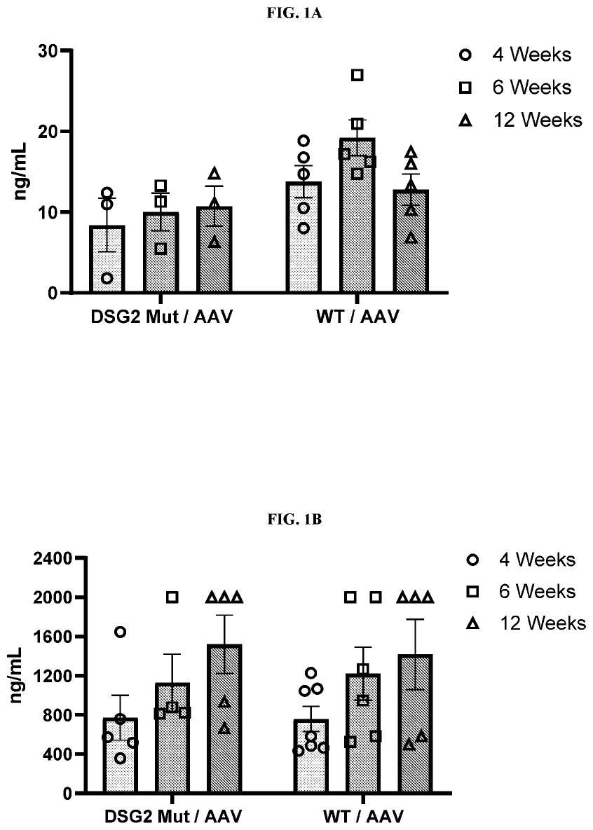

To investigate the expression level of sTGFβR2 and FGF21 in Dsg2^{mut/mut} mice, four-week-old mice were administered, via single intravenous retro-orbital or tail vein injection, rAAV-sTGFβR2 and rAAV-FGF21, each packaged in AAV8 capsid (AAV8-sTGFβR2 and AAV8-FGF21, respectively). AAV8-sTGFβR2 and AAV8-FGF21 were administered at dose of 3E12 vg/kg and 3E11 vg/kg, respectively. Control animals were administered vehicle only, sTGFβR2-Fc and FGF21 expression was measured by ELISAs using antibodies to mouse TGFβR2 and FGF21, respectively. As shown in FIG. 1A and FIG. 1B, the plasma levels of FGF21 and sTGFβR2 increased over time, respectively, in both Dsg2^{mut/mut} and wild-type mice administered AAV8-sTGFβR2 and AAV8-FGF21.

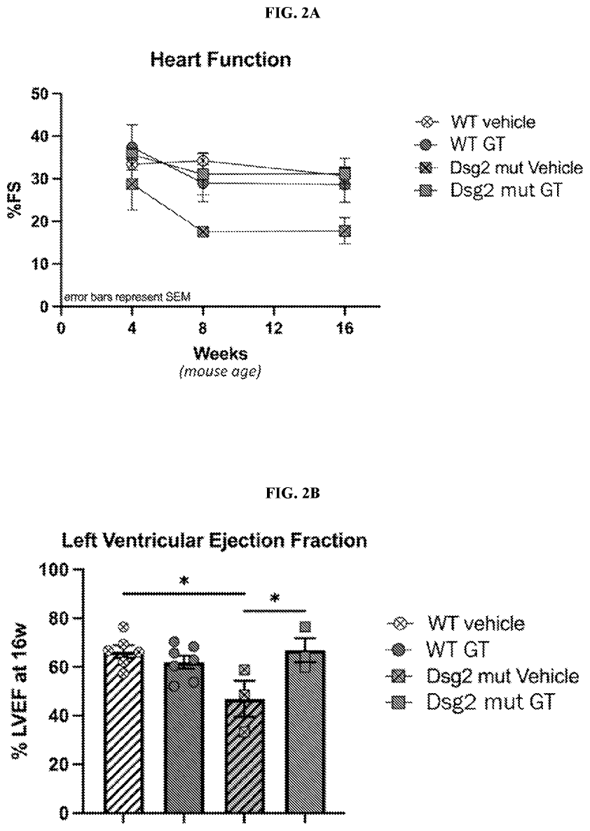

Fractional shortening (FS) and ejection fraction was measured by standard M-mode echocardiography. FS represents the percentage change in left ventricle (LV) chamber dimension and systolic contraction used to estimate LV wall contractility or the systolic function. FIG. 2A shows that heart function, as measured by percent fractional shortening (% FS), rapidly declines in control Dsg2^{mut/mut} mice administered vehicle. In comparison, % FS improved over time in Dsg2^{mut/mut} mice administered AAV8-sTGFβR2 and AAV8-FGF21. At 16 weeks of age, Dsg2^{mut/mut} mice administered vehicle exhibit a significant reduction in left ventricular ejection fraction (LVEF), as compared to Dsg2^{mut/mut} mice administered AAV8-sTGFβR2 and AAV8-FGF21 (FIG. 2B).

### Example 3: Expression of sTGFβR2 and FGF21 in Mouse Model of ARVC

PKP2cKO mice were generated as described in Cerrone et al. (2017) Nature Communications 8: 106. PKP2cKO mice have cardiomyocyte-specific, tamoxifen-induced knockout of the desmosomal protein plakophilin-2 (PKP2). PKP2 knockout induced PKP2cKO mice have previously been shown to develop progressive right-ventricular dominant arrhythmogenic cardiomyopathy. The general study design in this Example for using PKP2cKO mice to investigate the efficacy of sTGFβR2, FGF21, and bigenic sTGFJPR2/FGF21 gene therapy was to administer the gene therapy at 1.5E13 vg/kg to PKP2cKO mice (day −7), followed by tamoxifen-induced knockout seven days later (day 0). 21 days after knockout induction, the mice were subjected to isoproterenol (ISO) challenge and electrocardiography. 28 days after knockout induction, the mice were subjected to echocardiography and were then sacrificed and terminal blood was collected.

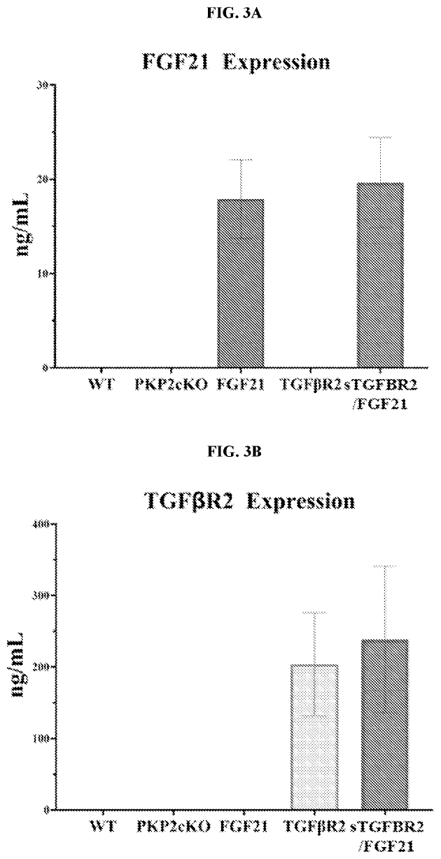

To investigate the expression level of sTGFβR2 and FGF21 in PKP2cKO mice, four-week-old mice were administered, via single intravenous retro-orbital or tail vein injection, rAAV-sTGFβR2 alone, rAAV-FGF21 alone, or a rAAV comprising an rAAV genome comprising a sTGFβR2-Fc coding sequence and a FGF21 coding sequence (“rAAV-sTGFβR2/FGF21”). The sTGFβR2, FGF21, and bigenic sTGFβR2/FGF21 vectors were each packaged in AAV8 capsid (AAV8-sTGFβR2, AAV8-FGF21, AAV8-sTGFβR2/FGF21, respectively). AAV8-sTGFβR2, AAV8-FGF21, and AAV8-sTGFβR2/FGF21 (or “bigenic gene therapy”) were each administered at a dose of 1.5E13 vg/kg. Control wild type (WT) and PKP2cKO animals were administered vehicle only, sTGFβR2-Fc and FGF21 expression was measured by ELISAs using antibodies to mouse TGFβR2 and FGF21, respectively. As shown in FIG. 3A and FIG. 3B, the expression levels of FGF21 and sTGFβR2 were confirmed in PKP2cKO mice administered AAV8-sTGFβR2, AAV8-FGF21, or AAV8-sTGFβR2/FGF21.

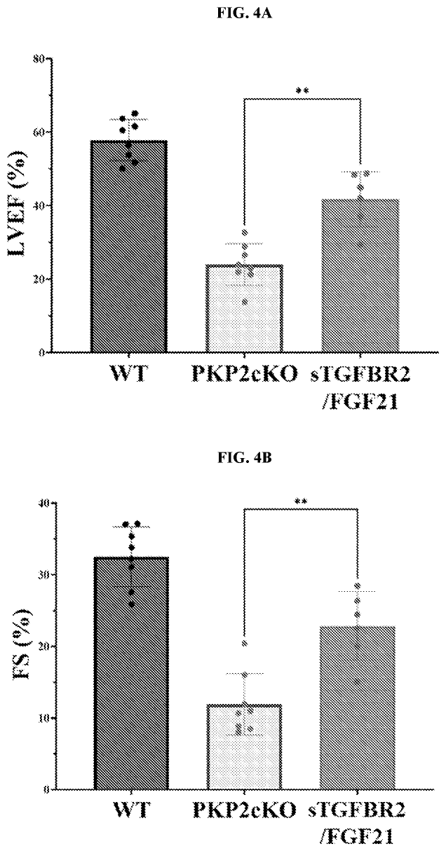

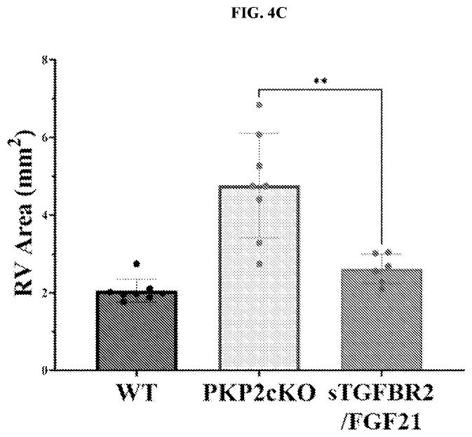

Cardiac structure and function of PKP2cKO mice treated with the combination gene therapy was assessed. Fractional shortening (FS) and ejection fraction was measured by standard M-mode echocardiography. As shown in FIGS. 4A-4C, administration of the bigenic gene therapy resulted in enhanced preservation of left ventricle systolic function measures as a function of left ventricle ejection fraction (LVEF: FIG. 4A), enhanced preservation of fractional shortening (FS; FIG. 4B), and significant reduction in right ventricle dilatation (FIG. 4C). Taken together, the administration of the bigenic gene therapy significantly improved cardiac structure and function of PKP2cKO mice.

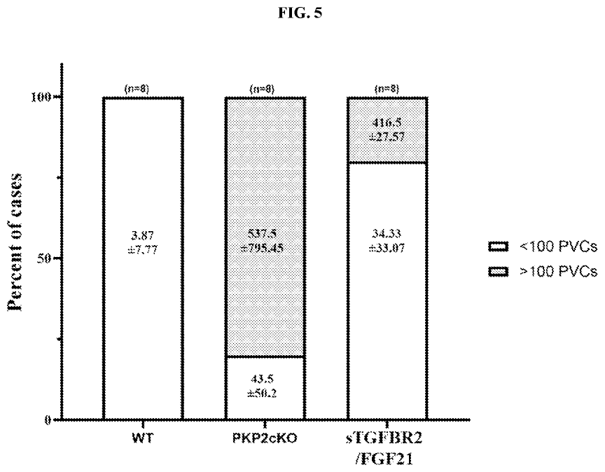

Arrhythmia burden was also assessed in PKP2cKO mice treated with the combination gene therapy. Arrhythmia burden, assessed by number of premature ventricular contractions (PVCs) is a clinically meaningful measurement in arrhythmogenic cardiomyopathy: it is a predictor of survival (correlates with mortality and life-threatening ventricular arrhythmias in arrhvthmogenic right ventricular cardiomyopathy, it is a key driver of symptoms, and it is directly linked to quality of life—even single PVCs can trigger anxiety and post-traumatic stress disorder in patients. As shown in FIG. 5, PKP2cKO mice administered the bigenic gene therapy were observed to have a reduction in the percentage of cases with PVCs greater than 100 in 30 minutes following ISO challenge.

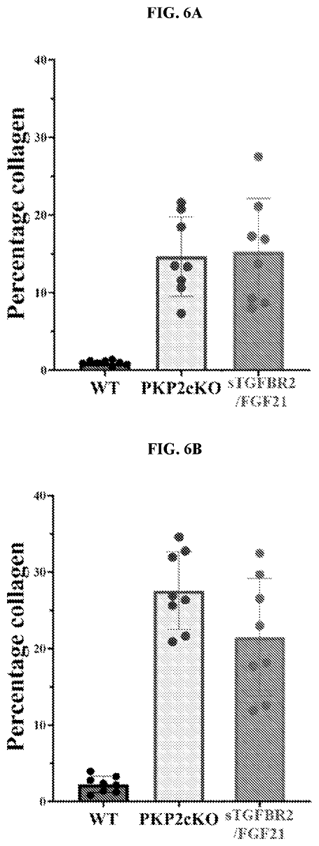

In order to investigate the progression of cardiac fibrosis, hearts of mice were obtained after sacrifice (28 days after induction of knockout), fixed, embedded, and sectioned according to standard techniques. Sections were then stained with Masson's Trichrome Staining and analyzed for cardiac fibrosis by assessing the percentage of collagen present, indicative of fibrosis. FIGS. 6A and 6B shows the level of fibrosis detected in the left ventricle (LV) and right ventricle (RV) of PKP2cKO hearts of mice that received the bigenic gene therapy or vehicle as control.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

We claim:

1. A method of treating an arrhythmogenic cardiomyopathy (ACM) in a subject, comprising administering to the subject a gene therapy comprising a first nucleic acid encoding soluble transforming growth factor beta receptor (sTGFβR2) and a second nucleic acid encoding fibroblast growth factor 21 (FGF21).

2. The method of claim 1, wherein the ACM is selected from the group consisting of arrhythmogenic right ventricular cardiomyopathy (ARVC), arrhythmogenic left ventricular cardiomyopathy (ALVC), and biventricular arrhythmogenic cardiomyopathy.

3. The method of claim 1 or 2, wherein the ACM is ARVC.

4. The method of any one of claims 1-3, wherein the subject is a mammal.

5. The method of any one of claims 1-4, wherein the subject is a human.

6. The method of any one of claims 1-5, further comprising determining a base level of fractional shortening (FS) and/or left ventricular ejection fraction (LVEF) in the subject prior to administration of the gene therapy.

7. The method of claim 6, wherein the treatment results in a reduction in the level of FS and/or LVEF in the subject at a duration after administration of the gene therapy, as compared to the base level of FS and/or LVEF in the subject.

8. The method of any one of claims 1-7, wherein the subject has arrhythmia.

9. The method of claim 8, further comprising determining a base level of arrhythmia in the subject prior to administration of the gene therapy.

10. The method of claim 8 or 9, wherein the treatment results in a reduction in the level of arrhythmia in the subject at a duration after administration of the gene therapy, as compared to the base level of arrhythmia in the subject.

11. The method of any one of claims 1-10, wherein the subject has been administered an effective amount of one or more therapeutic agents for the treatment of an ACM.

12. The method of any one of claims 1-10, further comprising administering to the subject an effective amount of one or more therapeutic agents for the treatment of an ACM.

13. The method of claim 11 or 12, wherein the one or more therapeutic agents for the treatment of an ACM is selected from the group consisting of an angiotensin-converting enzyme (ACE) inhibitor, an angiotensin II receptor blocker, an anti-arrhythmia agent, a beta blocker, a blood thinner, and a diuretic.

14. The method of any one of claims 1-13, wherein the gene therapy is administered intravenously.

15. The method of any one of claims 1-14, wherein the ACM is PKP2 mutation-associated ACM.

16. The method of any one of claims 1-15, wherein the ACM is DSG2 mutation-associated ACM.

17. The method of any one of claims 1-16, wherein the ACM is DSP mutation-associated ACM.

18. The method of any one of claims 1-17, wherein the ACM is DSC2 mutation-associated ACM.

19. The method of any one of claims 1-14, wherein the ACM is a genetically elusive ACM.

20. The method of any one of claims 1-19, wherein the first nucleic acid comprises a first transcriptional regulatory element operably linked to the sTGFBR2 coding sequence.

21. The method of any one of claims 1-20, wherein the sTGFBR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 1, 2, 3, 24, or 72.

22. The method any one of claims 1-21, wherein the sTGFBR2 coding sequence further comprises a heterologous or an innate secretion signal sequence, wherein the signal sequence is encoded by a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 4, 5, or 6.

23. The method of any one of claims 1-22, wherein the sTGFBR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 9, 10, 11, 12, 25, or 71.

24. The method of any one of claims 1-23, wherein the sTGFBR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 13, 14, 15, 26, or 74.

25. The method of claim 24, wherein the sTGFBR2 coding sequence further encodes a secretion signal sequence having at least 85%, 86%, 87%, 88%, 89%, 90%, 91%, 92%, 93%, 94%, 95%, 96%, 97%, 98%, 99%, or 100% sequence identity to SEQ ID NO: 7 or 8.

26. The method of claim 24 or 25, wherein the sTGFBR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, 17, 18, 19, 27, or 73.

27. The method of any one of claims 1-26 wherein the second nucleic acid comprises a second transcriptional regulatory element operably linked to the FGF21 coding sequence.

28. The method of any one of claims 1-27, wherein the FGF21 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 28, 29, 30, or 31.

29. The method any one of claims 1-28, wherein the FGF21 coding sequence encodes an amino acid sequence having at least 85%, 86%, 87%, 88%, 89%, 90%, 910%, 92%, 93%, 94%, 95%, 96%, 97%, 98%, 99%, or 100% sequence identity to SEQ ID NO: 32, 33, or 34.

30. The method of any one of claims 27-29, wherein the first and second transcriptional regulatory elements are each selected from the group consisting of one or more ApoE binding sites, an hAAT promoter, and an EF-1α promoter or variant thereof.

31. The method of any one of claims 27-30, wherein the first and second transcriptional regulatory elements each comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 34, 36, 38, 39, 40, and/or 48.

32. The method of any one of claims 1-31, wherein the first and second nucleic acid each further comprises a post-transcriptional regulatory element.

33. The method of claim 32, wherein the post-transcriptional regulatory element comprises a polyadenylation signal and/or WPRE sequence.

34. The method of claim 33, wherein the polyadenylation signal is an SV40 polyadenylation signal.

35. The method of claim 33, wherein the WPRE sequence is a WPRE3 sequence.

36. The method of any one of claims 32-35, wherein the post-transcriptional regulatory element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 50, 51, 70, or 75.

37. The method of any one of claims 1-36, wherein the first nucleic acid is comprised within a first vector, and the second nucleic acid is comprised within a second vector.

38. The method of claim 37, wherein the first vector and/or the second vector is each a viral vector, optionally wherein each is independently selected from the group consisting of adeno-associated virus (AAV), adenovirus, retrovirus, orthomyxovirus, paramyxovirus, papovavirus, picomavirus, lentivirus, herpes simplex virus, vaccinia virus, pox virus, and alphavirus.

39. The method of claim 37 or 38, wherein:
the first vector is an AAV vector comprised within a first recombinant AAV (rAAV), wherein the first rAAV comprises an AAV capsid comprising an AAV capsid protein; and a first rAAV genome; and/or
the second vector is an AAV vector comprised within a second rAAV, wherein the second rAAV comprises an AAV capsid comprising an AAV capsid protein; and a second rAAV genome.

40. The method of any one of claims 1-39, wherein the gene therapy comprises:
a first recombinant AAV (rAAV) comprising:
an AAV capsid comprising an AAV capsid protein; and
a first rAAV genome comprising a nucleic acid encoding the amino acid sequence set forth in SEQ ID NO: 26 or 73; and
a second rAAV comprising:
an AAV capsid comprising an AAV capsid protein; and
a second rAAV genome comprising a nucleic acid encoding the amino acid sequence set forth in SEQ ID NO: 33 or 34.

41. The method of claim 39 or 40, wherein the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 66 or 78.

42. The method of any one of claims 39-41, wherein the first rAAV genome further comprises a 5′ inverted terminal repeat (5′ ITR) nucleotide sequence, and a 3′ inverted terminal repeat (3′ ITR) nucleotide sequence.

43. The method of any one of claims 39-42, wherein the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77.

44. The method of any one of claims 39-43, wherein the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 67 or 79.

45. The method of any one of claims 39-44, wherein the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 68 or 80.

46. The method of any one of claims 39-45, wherein the second rAAV genome further comprises a 5′ inverted terminal repeat (5′ ITR) nucleotide sequence, and a 3′ inverted terminal repeat (3′ ITR) nucleotide sequence.

47. The method of claim 46, wherein the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77.

48. The method of any one of claims 39-47, wherein the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 69 or 81.

49. The method of any one of claims 39-48, wherein the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

50. The method of any one of claims 39-49, wherein the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

51. The method of any one of claims 1-36, wherein the first and the second nucleic acid are comprised within a vector, optionally wherein the first and the second nucleic acid are separated by a polycistronic element.

52. The method of claim 51, wherein the vector comprises:
the first nucleic acid comprising a nucleic acid encoding the amino acid sequence set forth in SEQ ID NO: 26 or 73; and
the second nucleic acid comprising a nucleic acid encoding the amino acid sequence set forth in SEQ ID NO: 33 or 34.

53. The method of claim 51 or 52, wherein the first and the second nucleic acid are separated by a polycistronic element.

54. The method of claim 53, wherein the polycistronic element is an IRES or 2A sequence.

55. The method of claim 53 or 54, wherein the polycistronic element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 55, 56, or 57.

56. The method of any one of claims 51-55, wherein the vector is an AAV vector comprised within a recombinant AAV (rAAV), wherein the rAAV comprises an AAV capsid comprising an AAV capsid protein; and a rAAV genome.

57. The method of claim 56, wherein the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

58. The method of claim 56 or 57, wherein the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.
